(19) United States
(12) Patent Application Publication
(10) Pub. No.: US 2005/0130161 A1
(43) Pub. Date: Jun. 16, 2005

(54) Tagging and recovery of elements associated with target molecules

(76) Inventors: Peter Fraser, Cambridge (GB); David Carter, Oxford (DE); Lyubomira Chakalova, Cambridge (GB)

Correspondence Address: FOLEY AND LARDNER;SUITE 500, 3000 K STREET NW, WASHINGTON, DC, 20007, US

(21) Appl. No.: 10/507,017

(22) PCT Filed: Mar. 7, 2003
(86) PCT No.: PCT/GB03/00984

(30) Foreign Application Priority Data
Mar. 8, 2002 (GB) 0205536.6
Aug. 5, 2002 (GB) 0218143/6

(51) Int. Cl. G06F 17/60; C12Q 1/68; G06F 19/00; G01N 33/48; G01N 33/50

(52) U.S. Cl. 435/6; 702/20; 705/3

(57) ABSTRACT

The invention provides a method for identifying elements associated with a target molecule comprising the steps of: (a) providing a probe capable of binding by specific molecular interaction to a predetermined specifically defined region of a target molecule, the probe associated with or capable of recruiting an enzyme; (b) adding a tag capable of being activated by the enzyme such that it can attach to elements in the vicinity of the enzyme; and (c) isolating elements having the tag attached thereto, wherein the defined region occurs once, twice, or in a low number of copies in the target molecule. Preferably the tag can attach only to elements in the vicinity of the enzyme.

The present invention relates to a new method for identifying elements associated with target molecules.

Many genes and gene clusters are controlled by known (or unknown) distant regulatory elements that are necessary for high-level expression. Identification of these regulatory elements is an expensive and time-consuming process. Previous attempts to identify such distant regulatory elements have used a number of different methods, but most directly by scanning large genomic regions for DNase I hypersensitivity sites, followed by functional analysis of those regions linked to reporter genes in transgenic mice. This method of identification will clearly take a very long time.

The beta-globin locus is the prototypical gene cluster regulated by distant regulatory elements; the search for the beta-globin regulatory elements took approximately 10 years. Experiments designed to locate the beta-globin gene regulatory elements began in the late 1970s. In the early 1980s data arose that suggested distant elements were involved. A thalassemia patient was studied whose genome contained an intact beta-globin gene but a large deletion upstream of the gene. This lead to the conclusion that a distant upstream element must be involved in the regulation of the gene (Kioussis et al., 1983). Indeed, transgenes containing the beta-globin gene alone achieve only very low levels of expression at best (Townes et al., 1985) In 1985 a series of DNase I hypersensitive sites were mapped 40-60 Kb upstream of the beta-globin gene (Tuan et al., 1985). In 1987 it was finally shown that this hypersensitive site region, collectively known as the locus control region (LCR), was sufficient to induce high level, position independent, copy number dependent gene expression when linked to the beta-globin gene (Grosveld et al., 1987). Defects in human beta-globin gene expression, or hemoglobinopathies, are the most common genetic diseases worldwide. The ability to induce high-level expression of an artificially introduced beta-globin gene is therefore of significant therapeutic use. In addition, the ability to locate control regions of other genes is clearly desirable.

Chromatin conformation capture (3C; Decker et al 2002) has been used to determine the conformation of a yeast chromosome to try to determine the interaction of genes and control regions. However, many technical problems arise when trying to apply this method to higher eukaryotes, not least because the mammalian genome is approximately 200 times the size of a yeast genome. The 3C has several disadvantages: 3C does not enable recovery of in situ labelled molecules, nor does 3C give a very high degree of resolution. In addition, other disadvantages of the 3C technique result because this technique allows only an average conformation of a chromosome to be calculated; this means that if all the cells used in the technique are not homogeneous or the molecular conformation is dynamic, specific interactions may be overlooked. Further, the 3C technique does not provide a method for determining which proteins or other molecules are associated with the genome.

Fluorescence in situ hybridisation (FISH) is a previously known techniques which uses hapten-labelled nucleotide probes followed by anti-hapten antibodies conjugated to fluorophores to determine the site of an actively transcribed gene via the antibody's ability to specifically bind to the hapten. Covalent tag deposition has commonly been used to enhance the signals obtained using the above technique. Kits enabling performance of covalent tag deposition to enhance signals are obtainable from NEN Dupont and are called TSA™ (Tyramide Signal Amplification™). However, this technique has not provided means for purifying molecular complexes from specific sites or in the immediate vicinity of specific sites in or on cells. Neither FISH nor TSA allow for detection (and thus identification) of, for example, the interaction of distant regulatory elements with an actively transcribed gene. There is no technique presently available to use for detecting(and thus identifying) the interaction of distant regulatory elements with an actively transcribed gene during the time of transcription.

Techniques are known which can be used for identification and analysis of proteins involved in protein complexes. ImmunoPrecipitation (IP) is most commonly used to ‘pull down’ proteins associated in a complex with a target protein(s). However no techniques exist to analyse, for instance, molecules or complexes which are only involved in “loose” functional interactions with another complex or which only function in the vicinity of another protein.

van Steensel et al(Nature Genetics, 27, 304-308, 2001) describe a method of genome-wide Chromatin profiling using targeted DNA adenine methyltransferase (DAM). A “GAGA factor” (GAF) conjugate with DAM binds predominantly to the motif GAGA, which motif is present in numerous euchromatic sites in chromosomes. This provided a large-scale technique for mapping of protein-binding sites in the genome of Drosophilia. Because methylation by tethered DAM spreads over 2-5 kb from a discrete protein binding sequence, target locus may be mapped with a resolution of a few kilobases.

According to the present invention there is provided a method for identifying elements associated with a target molecule comprising the steps of:
(a) providing a probe capable of binding by specific molecular interaction to a predetermined specifically defined region of a target molecule, the probe associated with or capable of recruiting an enzyme;
(b) adding a tag capable of being activated by the enzyme such that it can attach to elements in the vicinity of the enzyme; and
(c) isolating elements having the tag attached thereto,

wherein the defined region occurs once, twice, or in a low number of copies in the target molecule.

According to the invention it may be preferable that the tag can attach only to elements in the vicinity of the enzyme.

Further, according to the invention it may be that the “low copy number” of the defined region of the target molecule is selected from the group of integral numbers of more than 2 up to 1000.

The target molecules may include RNA molecules, DNA molecules, proteins or peptides, lipids, or other, artificial compounds.

The method of the invention differs significantly from that of van Steensel et al. Their method is used to modify DNA on a genome wide scale. By fusing the DAM methylase to a DNA-binding or chromatin protein, they aim to methylate DNA wherever the fusion protein interacts with genomic sequences. This may be hundreds to several tens of thousands (or even millions) of sites within an individual cells genome. They then recover a highly heterogenous, complex mixture of DNA molecules from an unknown number of unrelated genomic sites. The method of the invention on the other hand can be targeted to a single gene or DNA locus. Only genomic DNA sites in the immediate vicinity, or in contact with, the target locus are labelled and thus a much more specific mix of DNA molecules can be recovered. The van Steensel method is broadly targeted to a number of sites but the targets are unknown and unrelated. The method of the invention can specifically target a single site or sites, along with elements involved in functional interactions with that site.

It is a particular advantage of the present invention that it provides a method of using the precise targeting power of specific molecular interactions such as in situ hybridization or immunohistochemistry to bind a probe just to a specific or unique region of a target molecule such as a complementary DNA, genomic locus, RNA species, or a protein or lipid cellular structure, the probe associated with or capable of recruiting an enzyme. This allows tagging of elements associated with, and only in the vicinity of, that region of the target molecule.

When the target is RNA, the elements which may be associated with these target molecules and which may be identified (or whose mode of action can be understood) by using the technique of the present invention include: distant regulatory elements (i.e. DNA elements via their chromatin protein association) that are in proximity to the RNA of an actively transcribed gene; RNA binding proteins such as those involved in RNA processing or stabilization/regulation/etc; proteins and protein complexes which facilitate the interactions between regulatory elements and a gene; proteins and protein complexes involved in the activation of genes; proteins and protein complexes involved in the regulation of chromatin structure in and around active genes; and transcription factors.

When the target is DNA, the elements which may be associated with these target molecules and which may be identified (or whose mode of action can be understood) by using the technique of the present invention include: distant regulatory elements (i.e. DNA elements via their chromatin protein association) that are in proximity to the targeted DNA; other DNA elements in proximity to the targeted DNA, which may be for example, engaged in functional interactions with the target sequence (e.g. boundaries, insulators, structural or architectural interactions); analysis of higher order chromatin structure, for example the analysis of tertiary chromatin interactions (chromatin folding); mapping chromatin interactions in entire loci or whole genomes (with the aid of high throughput technology); protein/protein complexes involved in regulation of gene expression or the control of chromatin structure.

When the target is protein, the elements which may be associated with these target molecules and which may be identified (or whose mode of action can be understood) by using the technique of the present invention include: DNA elements in proximity to a protein; RNA molecules in proximity to a protein; or other proteins/protein complexes bound to, or in the vicinity of a targeted protein (e.g. identifying other protein components of the LCR-beta-globin gene complex at different stages of development, or identifying the in-vivo ligands of a specific receptor- or vice versa).

When the target is lipid, the elements which may be associated with these target molecules and which may be identified (or whose mode of action can be understood) by using the technique of the present invention include: DNA elements in proximity to a lipid or artificial compound RNA molecules in proximity to a lipid or artificial compound; or proteins/protein complexes bound to, or in the vicinity of a targeted lipid or artificial compound.

The probe usable in the present invention may be a DNA probe, an RNA probe or an antibody specific for a protein, lipid or other molecule.

The probes used can be associated with the enzyme through antibody/enzyme conjugates, or enzyme/target molecule fusion.

The method by which the enzyme may be targeted to a specific molecule may be varied depending on the molecule to be targeted. For example, using a labelled probe specific for a DNA molecule, using immuno-histochemistry, or using a fusion of a protein (or other molecule of interest) and the enzyme. Preferably antibody/enzyme conjugates may be used. In one preferred embodiment, when the target molecule is RNA, a hapten-labelled probe specific to the intron of an active gene can be added, followed by addition of a hapten-specific Fab fragment/enzyme conjugate. One hapten which may be used is digoxygenin (DIG); others include biotin, dinitriphenol and FITC.

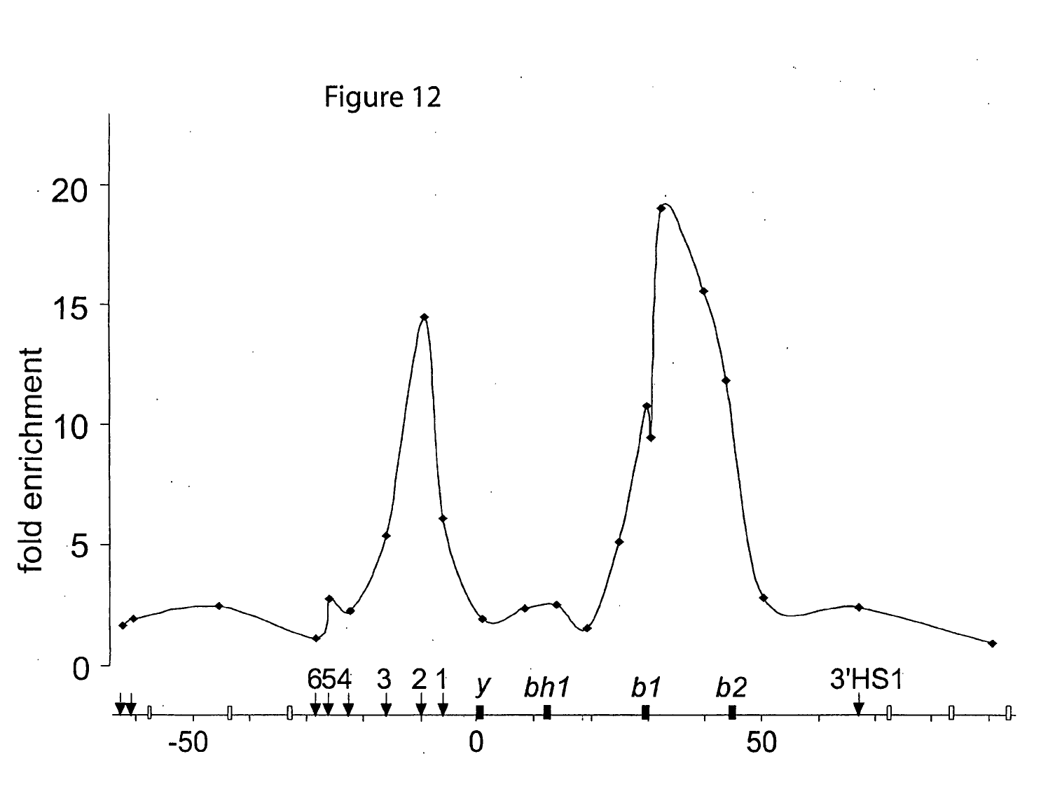

An enzyme which may be used in the present invention is Horse Radish Peroxidase. This enzyme can be used in combination with a tyramide molecule such as biotin-tyramide, dinitrophenol-tyramide or FITC-tyramide. These molecules form highly reactive, short-lived reactive radicals when catalysed by an enzyme, which bind to electron dense amino acids. As a result of their highly reactive nature, they only bind to amino acids in the immediate spatial vicinity. FIG. 12 shows a pronounced peak in the b1 and b2 loci, over a distance of 20-25 kb. The extent of the spread of these highly reactive radicals may be precisely controlled by varying the reaction conditions. This can result in a precise targeting method.

Another enzyme/TAG combination is ubiquitin-conjugating enzyme, with ubiquitin as a tag. Protein kinase could also be used as the enzyme (there are several with varied specificities) with phosphate as a tag. In this example a kinase which is able to add a phosphate to a nucleosomal protein (if looking for chromatin tagging) or other protein of interest should be used. Antibodies against the specifically modified epitope of the particular amino acid residue receiving the phosphate could be used to target isolate the tagged elements.

DNA Adenine Methyltransferase (DAM) is another enzyme which could be used, with a methyl group as the tag. In a slight variation of the procedure, instead of using a tag to pull out the labelled material one could use a restriction enzyme that will cut only DNA which is specifically methylated by DAM. DAM adds a methyl group to the adenine in the sequence GATC. This methylated site can only be cut by the DNA restriction endonuclease DpnI. DAM is normally only found in bacteria such as E. coli so it could be used in eukaryotic cells without any interference from endogenous methyltransferases which only methylate other sequence combinations. With this method no affinity chromatography is required. We would simply purify the DNA from the DAM treated cells and cut with DpnI and then isolate small DNA fragments that are released from the mixture of genomic DNA can be isolated. Careful selection of the target is preferred to prevent the DAM methylating sections of DNA, not in the immediate spatial vicinity of the interaction being studied. The small sites released by DpnI digestion can then be labelled with radioisotopes, etc., and used for diagnostic hybridization to a microarray, for example (van Steensel et al 2001).

Other enzyme/tag combinations could be used: any enzyme which can activate a tag molecule to deposit onto another molecule, for example protein, DNA, RNA, lipid etc in a manner such that the tagged product can then be isolated by whatever means (eg. affinity chromatography or immunoprecipitation) can be used in this technique.

Before separation, the molecules which have been tagged can be disrupted into smaller fragments using, for example, sonication, enzymatic cleaving, shearing with a French Press or small bore syringe, or another method which achieves such a result.

Analysis of the DNA obtained using the above method can be used to identify any regulatory elements which were in proximity to the active gene, because these elements become labelled with the tag, due to their proximity to the site HRP activity. The DNA can then be analysed by a number of quantitative techniques, for example Quantitative PCR (for example Real-Time PCR (Wittwer et al., 1997)) or semi-quantitative PCR, slot blot or microarray (Granjeaud et al., 1999), among others. This analysis allows scanning, high-throughput, high resolution analysis of any gene locus for hundreds or thousands of kilobases in either direction.

An embodiment of the present invention will now be described in more detail, by way of example, with reference to the drawings, in which:

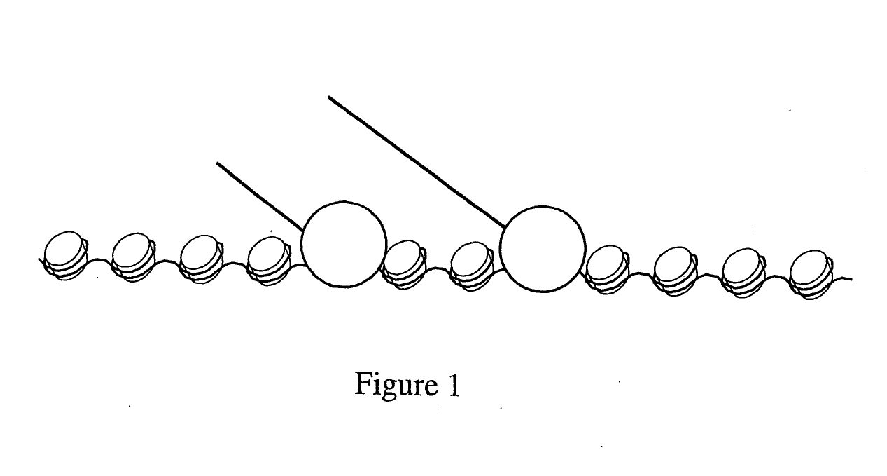

FIG. 1 is a schematic diagram showing a transcriptionally active gene in vivo. RNA polymerase II (open circles) transcribes a chromosomal gene or nucleosomal DNA template (DNA represented by curved lines wrapped around nucleosomes, (cylinders)). The RNA polymerase produces a nascent RNA primary transcript (diagonal straight lines).

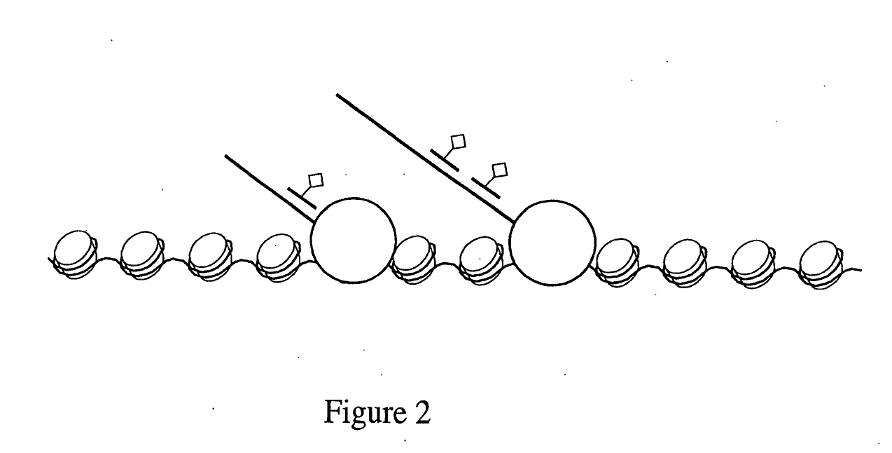

FIG. 2 is a schematic diagram showing in situ hybridisation. A complementary oligonucleotide probe is hybridised to the intron of the nascent RNA transcript. The oligonucleotide probe is labelled with a hapten, in this case digoxygenin (diamond).

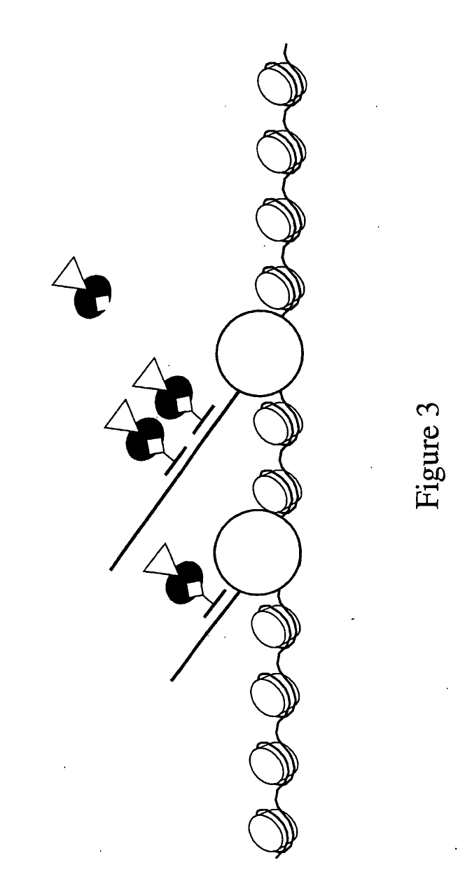

FIG. 3 is a schematic diagram showing immunological detection of hapten probe. An anti-digoxygenin antibody (black oval) conjugated to horse-radish peroxidase enzyme (triangle) is added. The antibody/peroxidase complex binds to the digoxygenin labelled, oligonucleotide probe.

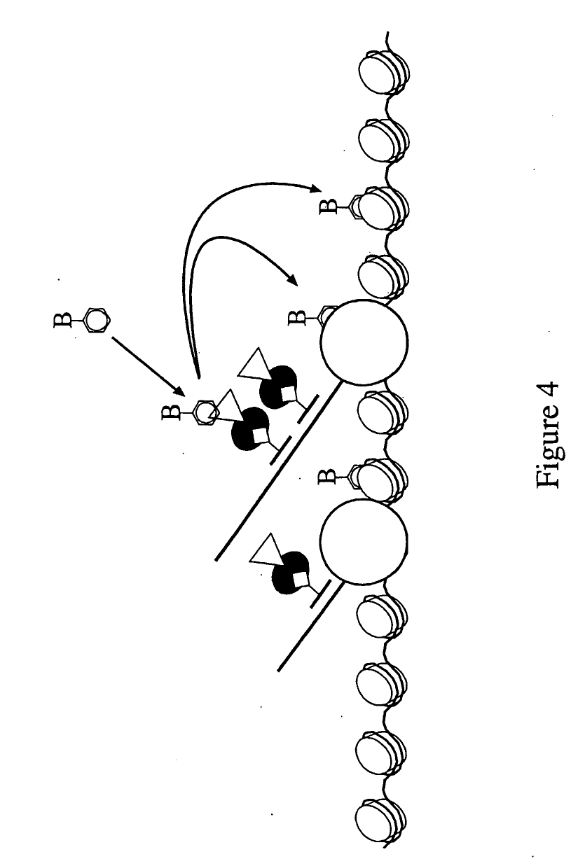

FIG. 4 is a schematic diagram showing the addition of biotin tyramide. Biotin-tyramide consists of a biotin molecule (B) linked to a phenol-like, tyramide chemical ring (hexagon with circle). When the tryamide comes in contact with the peroxidase, the tyramide is converted to a short-lived, highly reactive radical which is capable of immediate covalent attachment to electron dense moieties of nearby proteins.

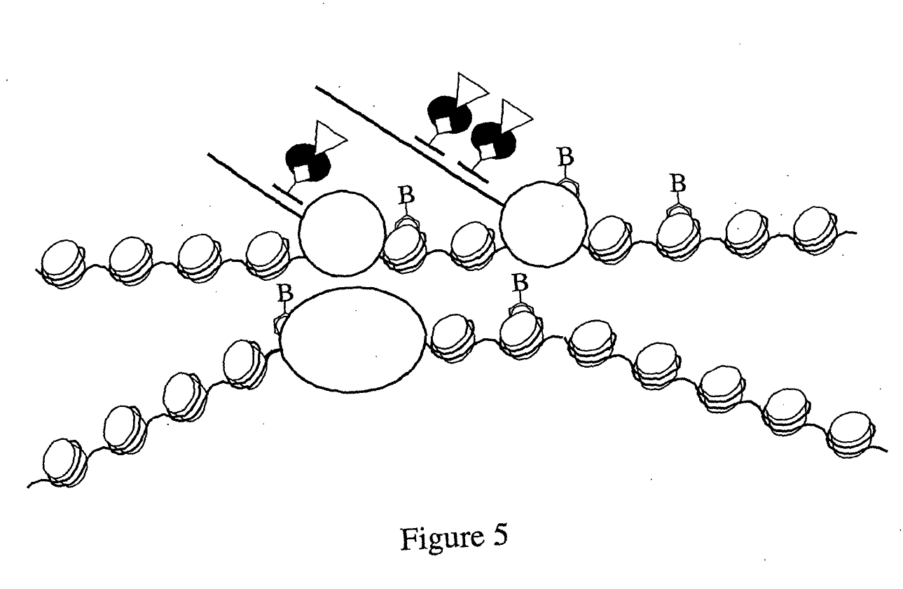

FIG. 5 is a schematic diagram showing the labelling of chromatin proteins in the immediate spatial vicinity. Biotin-tyramide deposition can also occur on chromatin proteins of sequences which are in the immediate vicinity. Such as, enhancers, locus control regions or other gene regulatory elements. DNA bound transcription factor (large oval).

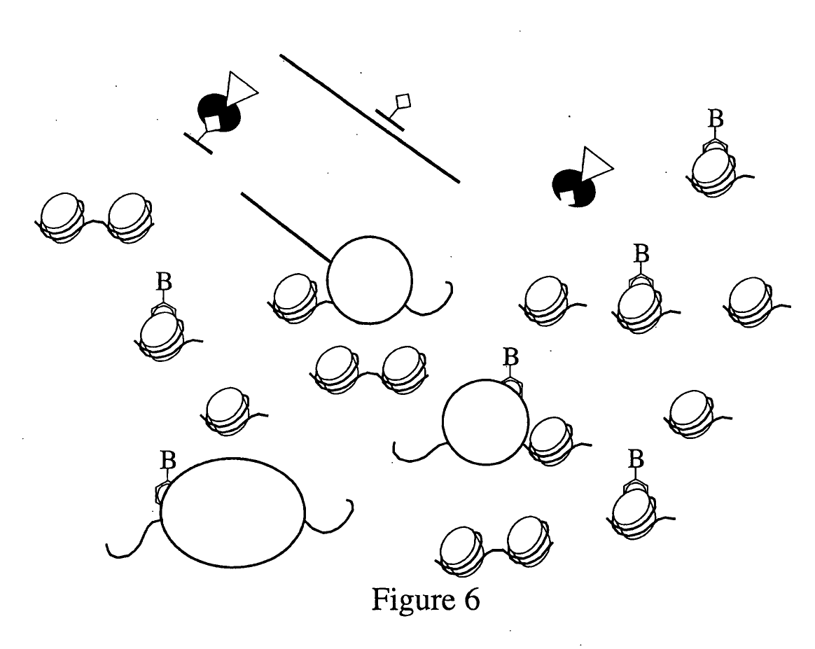

FIG. 6 is a schematic diagram showing the disruption of the chromatin. Chromatin is disrupted via sonication or some other method.

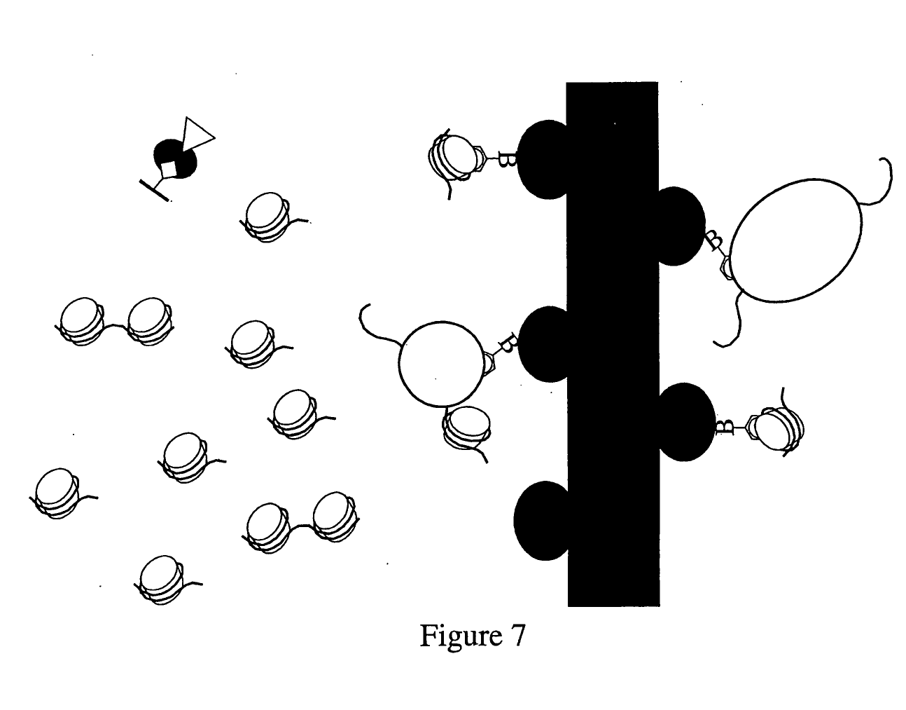

FIG. 7 is a schematic diagram showing purification of elements by affinity chromatography. Biotinylated protein/DNA complexes are purified by affinity chromatography with a strepavidin column.

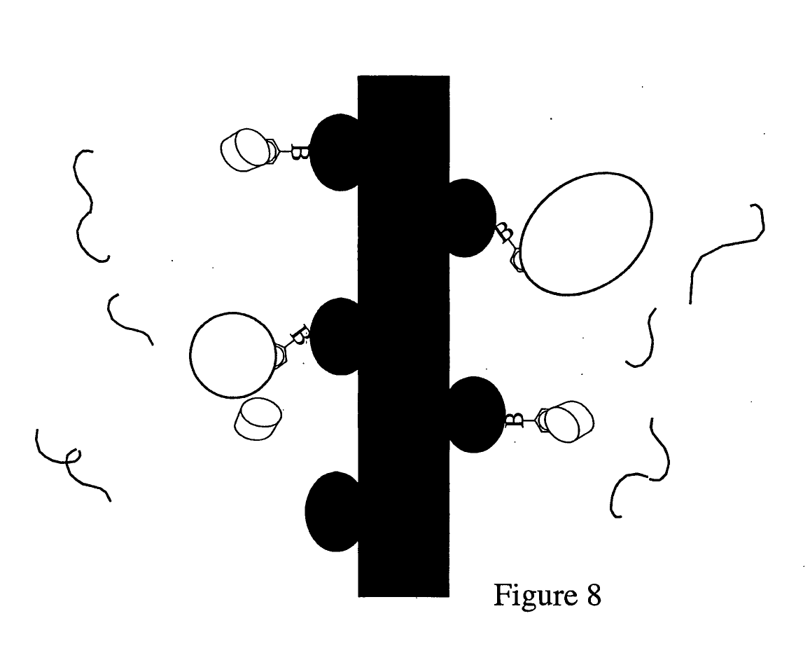

FIG. 8 is a schematic diagram showing cross link reversal. The formaldehyde chemical cross-links are reversed and DNA and/or proteins are purified for analysis.

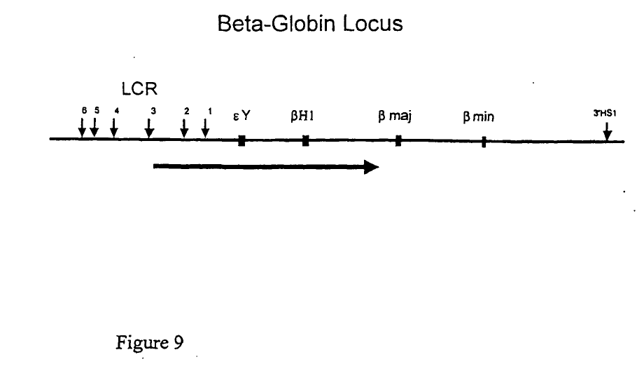

FIG. 9 is a schematic diagram showing the mouse beta-globin locus (genes=black boxes) and locus control region (LCR) and illustrates one model of LCR action: action at a distance.

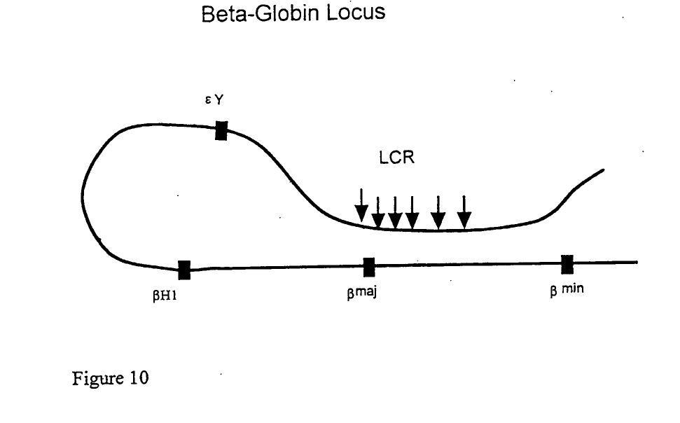

FIG. 10 is a schematic diagram showing the mouse beta-globin locus and locus control region (LCR) and illustrates another model of LCR action: direct LCR-gene interaction.

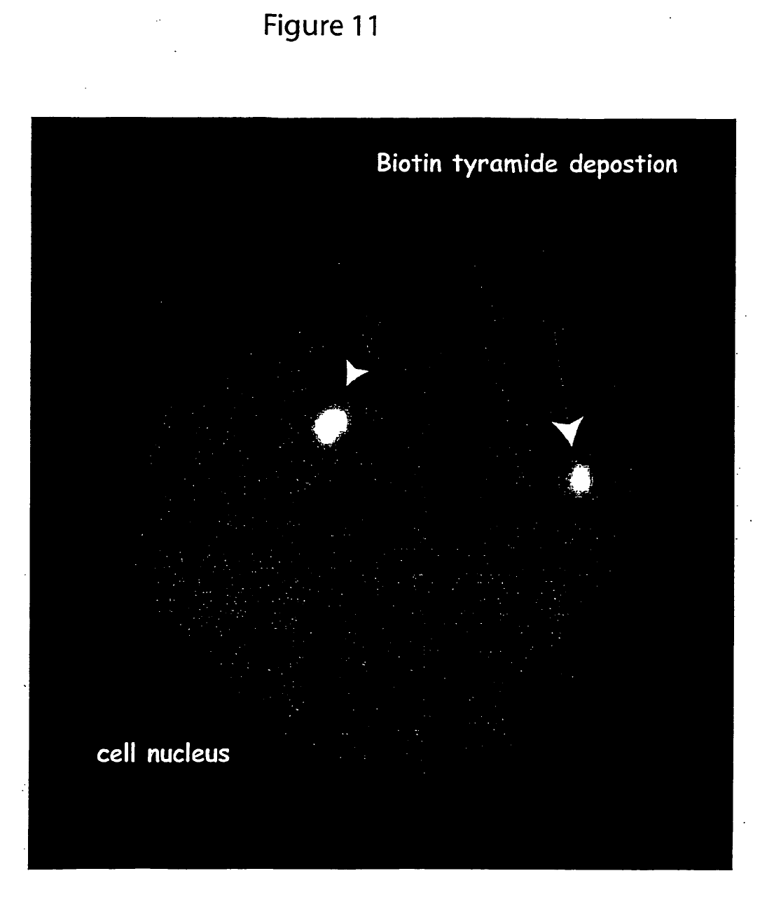

FIG. 11 is an image of a typical cell after visualisation of the specifically targeted biotin tyramide deposition.

FIG. 12 is a graph showing the results of Quantitative real-time PCR analyses of βmaj-directed RNA TRAP showing various sequences in the β globin locus and neighbouring olfactory receptor gene locus.

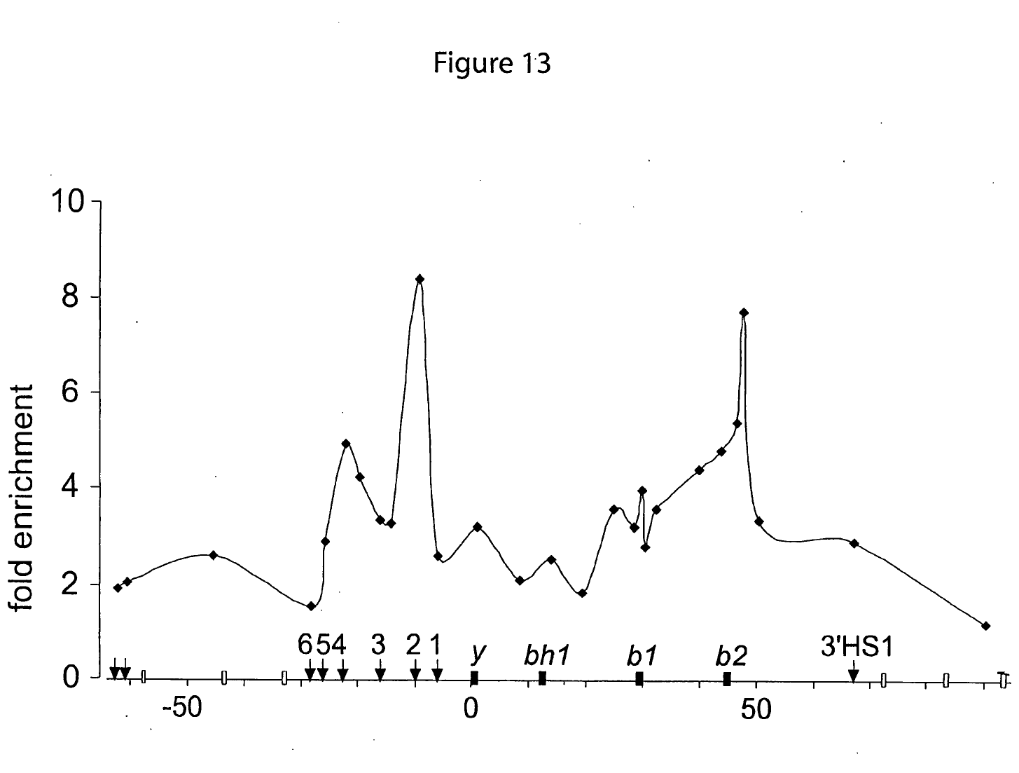

FIG. 13 is a graph showing the results of βmin-directed RNA TRAP assaying various sequences in the β globin locus and neighbouring olfactory receptor gene locus.

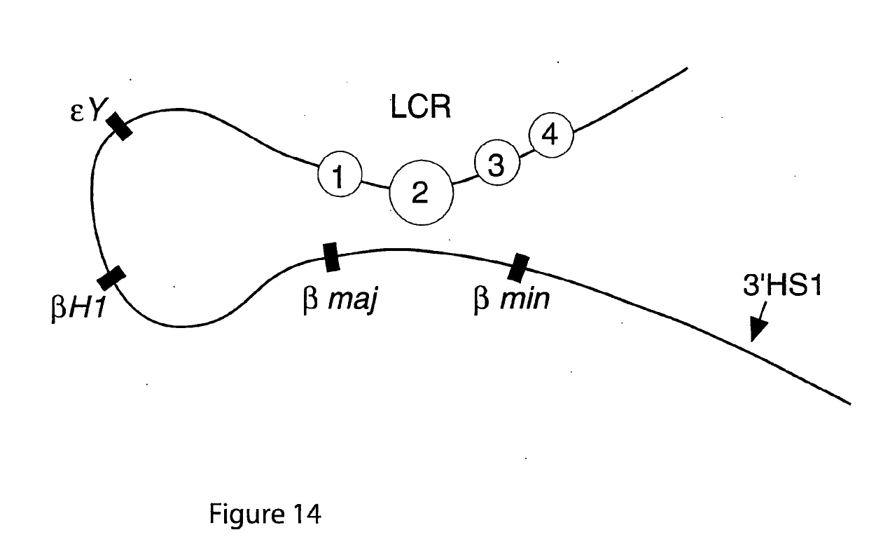

FIG. 14 is a schematic diagram showing the hypothesised interaction of the mouse beta-globin gene and locus control region (LCR).

Many genes and gene clusters are thought to be regulated by distant regulatory elements, which may be located tens to hundreds of kilobases away. The best characterised example of a distant element regulating a cluster of genes is the beta-globin locus control region (LCR), shown in FIG. 9. The LCR consists of a series of DNase I hypersensitive sites (HS) (1 to 6). At the core of each HS is a 200-300 bp region which is packed with transcription factor binding sites. The LCR is absolutely required for high level transcriptional activation of all the beta-globin genes. Two models have been proposed to explain the action of the LCR, although no direct proof exists for either mode of action, these are shown in FIGS. 9 and 10. The first model (FIG. 9) proposes that the LCR works at a distance. The LCR creates a large region of open chromatin surrounding the genes and recruits and sends factors necessary for gene activity along the chromatin. The second model (FIG. 10) proposes that the LCR physically contacts the gene(s) through long range chromatin interactions, essentially looping out the intervening sequences and activating transcription directly.

To determine if an actively transcribed beta-globin gene is in direct physical contact with the distant (40 Kb) LCR in vivo, the following technique was used (see FIGS. 1-8). Firstly, fetal liver, the main site of erythropoiesis in the developing foetus, is taken and disrupted, and the cells are spread in a monolayer on a slide, prior to cross-linking with formaldehyde. In situ hybridization is performed using a digoxygenin (DIG)-labelled oligonucleotide probe (FIG. 2), specific for the intron of the mouse beta-major globin gene. The enzyme Horse Radish Peroxidase (HRP) is then targeted to an RNA molecule using an anti-DIG antibody conjugated to Horse Radish Peroxidase (HRP) (FIG. 3), thus pinpointing HRP enzyme activity to the site of the actively transcribed gene.

Next, biotin-tyramide (FIG. 4) is added as a molecular tag; it is activated by the HRP to cause it to covalently attach to electron dense amino-acids in the immediate vicinity. After the tag is covalently attached (FIG. 5), the cells are sonicated to give small, soluble chromatin fragments (FIG. 6) having an average DNA size of 400 bp. The biotinylated chromatin is then purified using streptavidin agarose affinity chromatography (FIG. 7), cross-links are reversed and the DNA is purified. Multiple amplicons across the locus can then be analysed using quantitative or semi-quantitative PCR and/or slot blotting.

By using the above technique on the mouse beta-globin gene locus, it was found that high-level expression of the beta-globin genes is totally dependent on an extensively characterised, distal, regulatory element known as the LCR. The LCR and active beta-major gene are found to be in significant proximity in the mouse beta-globin locus in vivo; HS2 appears to be in intimate contact with the beta-major gene, and the two active adult genes also appear to be in close proximity (FIG. 3).

### EXAMPLES

### Example 1

RNA FISH-TRAP

E14.5d fetal livers from balb/c mice, in which only the adult-type b-maj and b-min genes are expressed, were disrupted in ice-cold PBS. The cells were spread on poly-L-lysine coated slides and fixed in 4% formaldehyde, 5% acetic acid for 18 minutes at room temperature. Subsequent slide-washing, permeabilization, probe-hybridisation, and post hybridisation washing were performed as described in Gribnau, J. et al. (1998); the probes used being directed to intron 2 near the 3′ ends of the mouse b-maj globin primary transcript. Endogenous peroxidases were quenched in 0.5% H₂O₂ (in PBS) for 10 minutes followed by washing (5 min) in TST (Tris, saline, Tween; 100 mM Tris ph7.5, 150 mMNaCl, 0.05% Tween 20) and blocking as described. Slides were then incubated with 1:100 dilution of anti-DIG fab fragment/HRP conjugate for 45 minutes at room temperature in a humidified chamber, washed twice (5 min each) in TST and then incubated for 1 minute with 1:150 biotin tyramide (NEN) under coverslips at room temp. The slides were then quenched again in 0.5% H₂O₂ (in PBS) for 10 minutes, washed twice in TST (5 min) and transferred to PBS ready for scraping. One of the slides was stained with an Avidin/Texas red conjugate for 45 minutes at room temperature. This slide was then washed, dehydrated, mounted and visualised as described in Gribnau, J. et al. (1998)

Cells were scraped from the remaining slides; typically approximately 25 million cells were recovered. The cells were spun down at 2900 g for 25 minutes, resuspended in 2M NaCl, 5M Urea, 10 mM EDTA, and sonicated for 200 seconds on ice (eight 25-second bursts with 1.5 minutes between bursts) using a Microson Ultrasonic cell Disruptor set at level 5. Crude chromatin was centrifuged for 15 minutes at 10,000 g, the supernatant containing the soluble chromatin was removed and the insoluble pellet was resuspended in 2M NaCl, 5M Urea, 10 mL EDTA, and sonicated again. The suspension was centrifuged again and the two soluble fractions were combined and dialysed overnight at 4° C. against PBS. This method routinely yielded chromatin fragments with an average DNA size of around 400 bp.

10% of the soluble chromatin was set aside as the input and the rest was passed over a streptavidin-agarose (Molecular Probes) affinity column. After binding, the column was washed with 3×700 μl PBS, 2×500 μl TSE 150 (20 mM Tris pH8.0, 1% Triton, 0.1% SDS, 2 mM EDTA, 150 mM NaCl), 2×500 μl TSE 500 (20 mM Tris pH8.0, 1% Triton, 0.1% SDS, 2 mM EDTA, 150 mM NaCl), and 3×700 μl PBS. The beads were then removed from the column, formaldehyde cross-links reversed and protein components digested by overnight incubation at 65° C. with 200 ug/ml proteinase K while shaking vigorously. The samples were treated with 20 Vg/ml RNase A for 30 min at 37° C., 200 μg/ml proteinase K for 5 hours at 37° C., phenol-extracted and ethanol-precipitated using 20 mg/ml glycogen as carrier. DNA from the input (IP) fraction was quantified using a standard spectrophotometer. DNA concentration of the affinity purified (AP) fraction was measured by picogren quantification using IP as a standard.

### Example 2

Real-Time PCR

Real-time PCR was performed with an ABI PRISM 7700 sequence detector using 2× SYBR green PCR master mix (Applied biosystems). For each primer pair a standard curve was generated using 30 ng, 5 ng, and 1 ng of IP which was then used to quantify the enrichment of 1 ng of AP (all reactions were performed in duplicate). All PCR products were run on a 2% agorose gel to ensure all reactions gave a single product.

Enrichment of various sequences across the β-globin locus and also across the neighbouring olfactory receptor gene (org), were measured using quantative real-time PCR. The measurements showed a 20-folded peak of enrichment near the transcription termination site of the b-maj gene, consistent with the position of the probes (FIG. 12). Enrichment dropped off sharply upstream of the b-maj gene for over 25 kb in the area of the developmentally silenced εy and βH1 genes, which are only sightly increased over background.

Strikingly, a peak of enrichment was observed over HS2, and to a lesser extent HS1 and HS3 of the LCR. This indicates these sites are in close association with the active gene.

The fact that other HS in the LCR (HS4, 5 and 6) and the downstream 3′HS1 (which is closer in base pairs to the βmaj gene than HS2) are not significantly enriched suggests they are outside the area of labelling and therefore not intimately associated with the active βmaj gene. Moreover, the low level of enrichment of these sites shows that there is no preferential labelling of areas of hypersensitive or open chromatin. To completely discount the possibility that these results were caused by a bias of biotin deposition in certain areas (e.g. open or hyper acetylated chromatin) a control random TRAP experiment was designed and performed. By omitting the intron probe during the FISH-stage, biotin deposition becomes random across the genome and therefore any bias for certain sequences would become apparent in the analysis of the AP material. There was no preferential selection for any of the sequences in the globin locus, thus verifying that enrichment of HS2 in the βmaj-directed TRAP experiment is due to proximity to the active βmaj gene and is not a chromatin bias. Repetition of the βmaj RNA TRAP assay three times obtained similar results. DNA from one of the βmaj RNA TRAP assays was analysed by slot blot with multiple probes yielding similar results. The data of this experiment provide the first direct evidence that a distal enhancer is held in significant physical proximity to an active gene that it regulates in vivo.

To distinguish between a co-transcriptional model in which both genes share the LCR simultaneously or an alternating model in which the LCR is involved exclusively with a single active gene. RNA-TRAP was repeated using intron probes to the βmin gene located approximately 15 kb downstream of βmaj. The results of this showed that HS2 is highly enriched in the βmin-directed AP chromatin, indicating it is tightly associated with the active βmin gene (FIG. 13). In addition, HS4 of the LCR was significantly enriched over background levels and when compared to HS1, 3, 5 and 6 of the LCR. The high level of enrichment of HS2 in both the βmin and βmaj directed RNA-TRAP assays indicates it is tightly associated with the active gene for most of the time primary transcript is present. The fact that βmaj-TRAP does not bring down the βmin gene and vice versa indicates the two genes are not closely associated.

There are many applications for the technique of the present invention, which can be performed in vivo, ex vivo, or in vitro.

One example of such a use is in transgenic animal technology: transgenic animals are presently being used by a number of laboratory around the world as bioreactors to produce large amounts of proteins of interest. The most commonly used method is to express the protein of interest in milk under control of a highly expressed milk protein gene promoter. Most transgenic animals created with such a construct would not express the protein or express it at very low levels making them unusable. Some transgenic animals may, by virtue of position effects at the site of integration of the construct, express larger amounts of the protein of interest. The addition of milk protein gene LCR-like sequences to the expression construct would increase the number of transgenic animals which express the gene to 100% and increase the average level of expression in every animal. This would significantly decrease the cost of production and greatly increase the yield.

When RNA is the target molecule, the method of the present invention labels only the cells in the population that are actively transcribing the gene of interest. The advantage of this is specifically interacting sequences are highly enriched upon affinity chromatography, whether the population is heterogeneous or the interaction is dynamic (Wijgerde et al., 1995). Another advantage of the present invention when RNA is the target molecule is this technique can detect (and thus identify) the interaction of distant regulatory elements with an actively transcribed gene during the time of transcription. There is no other technique we know of which can be used for this purpose. This technique can specifically label and recover proteins at the site of transcription in a dynamic or heterogeneous population of cells and identify specific interactions.

Another advantage of the present invention which results whatever the target molecule is, is the possibility of labelling and recovering complexes in the vicinity of a target complex (as opposed to molecules which are in direct interaction). The resultant enriched proteins could be analysed by a number of protein chemistry techniques such as Western blotting, Mass Spectroscopy, fractionation, purification, polyacrylamide gel electrophoresis, etc.

The present invention provides a relatively easy and rapid method which can detect interactions between an actively transcribed gene and distant regulatory element(s). The technique can also be used to identify any sequence element involved in an interaction with any other target sequence in vivo by virtue of their proximity.

The present invention provides a new way to identify the regulatory elements involved in the activation of genes in a rapid and relatively inexpensive way. It has also been used to address the question of how LCRs or enhancer elements function and in fact has provided the first direct evidence that the LCR functions by physically interacting with an actively transcribed gene in the beta-globin locus.

Data with RNA FISH shows that the method of the invention has clearly identified HS2 of the beta-globin locus control region. HS2 has been shown previously through functional studies to be major, classical enhancer element of the locus control region that drives beta-globin gene expression in vivo. Therefore in similar experiments with other genes the major enhancer element(s) driving those genes could be identified by this technique. Function and/or industrial applications of the isolated elements could be inferred.

### REFERENCES

Bobrow M., Harris, T., Shaughnessy,K. and Litt, G. Catalyzed reporter deposition, a novel method of signal amplification—application to immunoassays. Journal of Immunological Methods 125: 279-285 1989

Dekker, J., Rippe, K., Dekker, M., and Kleckner, N. (2002). Capturing chromosome conformation. Science 295, 1306-11.

Granjeaud, S., Bertucci, F., and Jordan, B. R. (1999). Expression profiling: DNA arrays in many guises. Bioessays 21, 781-90.

Grosveld, F., van Assendelft, G. B., Greaves, D. R., and Kollias, G. (1987). Position-independent, high-level expression of the human beta-globin gene in transgenic mice. Cell 51, 975-85.

Kioussis, D., Vanin, E., deLange, T., Flavell, R. A., and Grosveld, F. G. (1983). Beta-globin gene inactivation by DNA translocation in gamma beta- thalassaemia. Nature 306, 662-6.

Townes, T. M., Lingrel, J. B., Chen, H. Y., Brinster, R. L., and Palmiter, R. D. (1985). Erythroid-specific expression of human beta-globin genes in transgenic mice. Embo J 4, 1715-23.

Tuan, D., Solomon, W., Li, Q., and London, I. M. (1985). The “beta-like-globin” gene domain in human erythroid cells. Proc Natl Acad Sci U S A 82, 6384-8.

van Steensel, B., Delrow, J. and Henikoff, S. Chromatin profiling using targeted DNA adenine methyltransferase. Nature Genetics Volume 27 Mar. 2001

Wijgerde, M., Grosveld, F., and Fraser, P. (1995). Transcription complex stability and chromatin dynamics in vivo. Nature 377, 209-13.

Wittwer, C. T., Herrmann, M. G., Moss, A. A., and Rasmussen, R. P. (1997). Continuous fluorescence monitoring of rapid cycle DNA amplification. Biotechniques 22, 130-1, 134-8.

1. A method for identifying elements associated with a target molecule comprising the steps of:
(a) providing a probe capable of binding by specific molecular interaction to a predetermined specifically defined region of a target molecule, the probe associated with or capable of recruiting an enzyme;
(b) adding a tag capable of being activated by the enzyme such that it can attach to elements in the vicinity of the enzyme; and
(c) isolating elements having the tag attached thereto,
wherein the defined region occurs once, twice, or in a low number of copies in the target molecule.

2. A method according to claim 1 wherein the tag can attach only to elements in the vicinity of the enzyme.

3. A method according to claim 1 wherein the low copy number of the defined region of the target molecule is selected from the group of integral numbers of more than 2 up to 1000.

4. A method according to claim 1, in which the target molecule is selected from the group consisting of RNA molecules, and DNA molecules.

5. A method according to claim 1, in which the target molecule is selected from the group consisting of proteins or peptides, lipids, or other, artificial compounds.

6. A method according to claim 1 in which the elements which may be associated with the target molecule include distant regulatory elements, RNA, DNA, proteins and protein complexes, transcription factors, or in-vivo ligands of a specific receptor.

7. A method according to claim 4 in which the probe is selected from the group consisting of DNA probe, and an RNA probe.

8. A method according to claim 5 in which the probe is selected from the group consisting of an antibody specific for a protein, lipid or other molecule.

9. A method according to claim 1 in which the probe is associated with the enzyme through an antibody/enzyme conjugate, or enzyme/target molecule fusion.

10. The method according to claim 1 in which the enzyme is targeted using a hapten labelled probe and then a hapten-specific Fab fragment-enzyme conjugate is added.

11. The method according to claim 1 in which the enzyme is targeted to RNA using a hapten-labelled probe specific to the RNA of an intron of an active gene, and then a hapten-specific Fab fragment/enzyme conjugate is added.

12. The method according to claim 10 in which the hapten is dioxygenin, biotin, dinitrophenol or FITC.

13. The method according to claim 1 in which the enzyme is Horse Radish Peroxidase and the tag is biotin-tyramide.

14. The method according to claim 1 in which elements are isolated using affinity chromatography or ImmunoPrecipitation.

15. A method for identifying elements of chromatin associated with transcribing RNA comprising the steps of:
(a) providing a hapten-labelled probe capable of binding by specific molecular interaction to a predetermined specifically defined region of RNA of a gene,
(b) providing an antibody conjugated with the enzyme horse-radish peroxidase, the antibody being specific for the hapten;
(c) adding biotin-tyramide by such that it can attach to elements in the vicinity of the enzyme;
(d) disrupting the chromatin; and
(e) isolating elements of chromatin having biotin attached thereto using affinity chromatography and purifying the elements.

16. The method according to claim 15 wherein in step (c) the tag can attach only to elements in the vicinity of the enzyme.

17. The method of claim 15 in which the chromatin is disrupted using sonication, enzymatic cleaving, or shearing with a French Press or small bore syringe.

18. The method according to claim 15 in which the hapten is digoxygenin.

19. Elements isolated by the method of any preceding claim 1.

20. A method for identifying DNA associated with a target molecule comprising the steps of:
(a) providing a probe capable of binding by specific molecular interaction to a predetermined specifically defined region of a target molecule, the probe associated with an DNA Adenine Methyltransferase;
(b) adding a restriction enzyme that will cut only DNA specifically methylated by DAM;
(c) isolating DNA cut by the restriction enzyme; and
(d) identifying the isolated DNA.

21. The method according to claim 20 wherein the isolated DNA is analysed/identified using Quantitative Real-Time PCR, slot blot or microarray.

22. A method for conducting a drug discovery business, comprising:
(i) by the method of claim 1, identifying DNA and/or protein associated with regulating gene expression;
(ii) generating a drug screening assay for identifying agents which inhibit or potentiate regulation of gene expression by the DNA and/or protein identified in step (i);
(iii) conducting animal toxicity profiles on an agent identified in step (ii), or an analogue thereof;
(iv) manufacturing a pharmaceutical preparation of an agent having a suitable animal toxicity profile; and
(v) marketing the pharmaceutical preparation to healthcare providers.

23. A method for conducting a bioinformatics business, comprising:
(i) by the method of claim 1, identifying DNA and/or protein associated with a gene at a chromosome location under a given condition; and repeating step (i); thereby
(ii) generating a database comprising information identifying different DNA and/or protein associated with one or more genes under one or more conditions.
